(19) United States
(12) Patent Application Publication
(10) Pub. No.: US 2014/0127142 A1
(43) Pub. Date: May 8, 2014

(54) TOOTHPASTE COMPOSITION FOR DENTIN HYPERSENSITIVITY

(71) Applicants: Noritaka Takahashi, Koto-ku (JP); Atsushi Yamagishi, Funabashi-shi (JP)

(72) Inventors: Noritaka Takahashi, Koto-ku (JP); Atsushi Yamagishi, Funabashi-shi (JP)

(73) Assignee: KAO CORPORATION, Chuo-ku (JP)

(21) Appl. No.: 14/126,303

(22) PCT Filed: Jun. 12, 2012
(86) PCT No.: PCT/JP2012/065027

§ 371 Date: Dec. 13, 2013

(30) Foreign Application Priority Data
Jun. 13, 2011 (JP) 2011-131698

(51) Int. Cl. A61K 8/58; A61Q 11/00

(52) U.S. Cl. CPC A61K 8/58; A61Q 11/00
USPC 424/49

(57) ABSTRACT

Provided is a toothpaste composition for dentin hypersensitivity that effectively prevents pain attributed to dentin hypersensitivity, instantaneously and sufficiently seals the openings of dentinal tubules in an exposed dentinal surface, and has excellent sustainability of the effect of suppressing the pain attributed to dentin hypersensitivity. The toothpaste composition for dentin hypersensitivity comprises the following components (A), (B), and (C): (A) a powder having a sparingly water-soluble property selected from a fatty acid divalent metal salt (A-1) derived from a saturated fatty acid having from 12 to 18 carbon atoms, a fatty acid acyllysine (A-2) having an acyl group derived from a saturated fatty acid having from 12 to 18 carbon atoms, and a fatty acid acyltaurine divalent metal salt (A-3) having an acyl group derived from a saturated fatty acid having from 12 to 18 carbon atoms; (B) a desensitizing agent selected from potassium nitrate, potassium citrate, strontium chloride, and strontium acetate; and (C) water; in which a mass ratio ((A)/(B)) of the component (A) to the component (B) is from 0.02 to 0.5.

### FIELD OF THE INVENTION

The present invention relates to a toothpaste composition for dentin hypersensitivity containing a powder having a sparingly water-soluble property.

### BACKGROUND OF THE INVENTION

Dentin hypersensitivity is a disease that causes pain when a mechanical, thermal, or chemical stimulus is applied to a tooth surface. Progression of periodontitis, aging, forceful tooth brushing, or the like causes gingival recession and damage to the dental enamel in a tooth. As a result, the dentin covered by the gingiva and dental enamel in the tooth is exposed, thereby causing the disease. Hitherto, various proposals have been made for prevention or treatment of such dentin hypersensitivity.

For example, Patent Document 1 discloses a method involving applying an effective dose of a potassium salt selected from potassium bicarbonate and potassium chloride. The method is intended to reduce the activity of a sensory nerve itself by an action of potassium ions, thereby alleviating pain. Further, Patent Document 2 discloses not only a soluble potassium salt such as potassium nitrate but also a soluble stannous salt and a soluble strontium salt such as strontium chloride as active components that are capable of alleviating sensitivity of dentin.

Meanwhile, promotion of remineralization of a damaged tooth to recover the original healthy state thereof can contribute to suppression of the onset of dentin hypersensitivity. For example, Patent Document 3 discloses a composition for an oral cavity containing a fluorine compound and a zinc compound having a sparingly water-soluble property, such as zinc oxide, zinc citrate, or zinc stearate, as components for remineralization.

In the meantime, the hydrodynamic theory is supported as a mechanism of the onset of dentin hypersensitivity, the hydrodynamic theory being that dentinal tubules are open in an exposed dentinal surface, external stimuli are transmitted from each opening to a tissue fluid in each of the dentinal tubules, a flow of the tissue fluid arrives at the dental pulp nerve located at the deep end portion in each of the tubules, and pain is perceived. In order to suppress the onset of dentin hypersensitivity on the basis of the theory, for example, Patent Document 4 discloses an embrocation for dentin hypersensitivity having such a construction that a first liquid containing an aluminum compound and an aid containing a phosphate compound or the like are separated from each other. The embrocation is used for attempting to seal physically openings of dentinal tubules by reacting the first liquid and the aid, thereby depositing an aluminum salt therein. Further, the preventive and therapeutic agent for dentin hypersensitivity disclosed in Patent Document 5 aims at sealing openings of dentinal tubules by using a colloid formed by a reaction of a metal salt such as aluminum lactate with, for example, a polyol phosphate ester, as the composition for oral cavity disclosed in Patent Document 3 does.

### CITATION LIST

### Patent Document

[Patent Document 1] JP-A-61-501389

[Patent Document 2] JP-A-2000-281551

[Patent Document 3] JP-A-2000-247852

[Patent Document 4] JP-A-6-116153

[Patent Document 5] JP-A-5-117157

### SUMMARY OF THE INVENTION

The present invention provides a toothpaste composition for dentin hypersensitivity, comprising the following components (A), (B), and (C):

(A) a powder having a sparingly water-soluble property selected from a fatty acid divalent metal salt (A-1) derived from a saturated fatty acid having from 12 to 18 carbon atoms, a fatty acid acyllysine (A-2) having an acyl group derived from a saturated fatty acid having from 12 to 18 carbon atoms, and a fatty acid acyltaurine divalent metal salt (A-3) having an acyl group derived from a saturated fatty acid having from 12 to 18 carbon atoms;

(B) a desensitizing agent selected from potassium nitrate, potassium citrate, strontium chloride, and strontium acetate; and

(C) water;

in which a mass ratio ((A)/(B)) of the component (A) to the component (B) is from 0.02 to 0.5.

### BRIEF DESCRIPTION OF THE DRAWINGS

FIG. 1(a) is a micrograph showing a dentinal surface that has dentinal tubules almost all of whose openings are exposed to an external environment, FIG. 1(b) is a micrograph showing a dentinal surface that has dentinal tubules wherein almost all of their openings are sealed, and FIG. 1(c) is a micrograph showing a dentinal surface that has dentinal tubules wherein some parts of openings are open but a majority of their openings are sealed.

### DETAILED DESCRIPTION OF THE INVENTION

Each component disclosed in Patent Document 1 or 2 reduces the activity of a sensory nerve and contributes to changing the balance of ions existing in the surrounding area of the sensory nerve, thereby making the sensory neuron insensitive. Accordingly, the component not only is unable to reduce pain sufficiently but also lacks an instantaneity and sustainability for alleviating pain. Further, the remineralization caused by the composition for oral cavity disclosed in Patent Document 3 is a mild action, and hence suppressing the onset of dentin hypersensitivity instantaneously and sustaining the effect are both difficult.

Thus, it is proposed, as means for preventing the manifestation of hypersensitive dentin, to seal the openings of dentinal tubules, thereby suppressing the flow of a tissue fluid in the dentinal tubule, by using, for example, the agents disclosed in Patent Documents 4 and 5. However, an aluminum lactate salt is formed in Patent Document 4, and hence it is difficult to allow the salt to deposit effectively into the openings of dentinal tubules. Further, it is also difficult to seal the openings of dentinal tubules sufficiently by using the colloid disclosed in Patent Document 5. As mentioned above, the agents disclosed in all the patent documents lack an instantaneity and sustainability for alleviating pain attributed to dentin hypersensitivity.

Therefore, the present invention relates to providing a toothpaste composition for dentin hypersensitivity that effectively prevents pain attributed to dentin hypersensitivity, instantaneously and sufficiently seals the openings of dentinal tubules on the exposed dentinal surface, and has excellent sustainability of an effect of suppressing the pain attributed to dentin hypersensitivity.

The inventors of the present invention made intensive studies in view of the above-mentioned problems. As a result, the inventors found that it is possible to provide a toothpaste composition for dentin hypersensitivity by combining a powder having a sparingly water-soluble property selected from a specific fatty acid divalent metal salt, fatty acid acyllysine, and fatty acid acyltaurine divalent metal salt, and a desensitizing agent selected from potassium nitrate, potassium citrate, strontium chloride, and strontium acetate, together with water, the toothpaste composition being capable of effectively preventing pain attributed to dentin hypersensitivity, instantaneously and sufficiently sealing the openings of dentinal tubules on an exposed dentinal surface, and sustainably suppressing the pain attributed to dentin hypersensitivity.

According to the toothpaste composition for dentin hypersensitivity of the present invention, it is possible to effectively prevent pain attributed to dentin hypersensitivity, to instantaneously and sufficiently seal the openings of dentinal tubules on an exposed dentinal surface, and to sustain the effect of suppressing the pain attributed to dentin hypersensitivity.

The toothpaste composition for dentin hypersensitivity of the present invention contains a powder having a sparingly water-soluble property (A) selected from a fatty acid divalent metal salt (A-1)derived from a saturated fatty acid having from 12 to 18 carbon atoms, a fatty acid acyllysine (A-2) having an acyl group derived from a saturated fatty acid having from 12 to 18 carbon atoms, and a fatty acid acyltaurine divalent metal salt (A-3) having an acyl group derived from a saturated fatty acid having from 12 to 18 carbon atoms. The powder having a sparingly water-soluble property (A) used in the present invention is a powder that dissolves in water at 25° C. in an amount of less than 0.1 g with respect to 100 g of the water, and preferably is a powder that dissolves in water at 25° C. in an amount of less than 0.05 g with respect to 100 g of the water or powder that is substantially insoluble in water. Further, the powder having a sparingly water-soluble property (A) is excellent in spreadability, and hence each bristle tip of a toothbrush or the like comes into contact with an exposed dentinal surface while applying a burden thereto directly or indirectly at the time of brushing. Accordingly, the openings of dentinal tubules can be sealed with the powder having a sparingly water-soluble property (A) which is contained in the toothpaste composition. The powder having a sparingly water-soluble property (A) spreads well, that is, has good spreadability, and hence, when the toothpaste composition for dentin hypersensitivity of the present invention is used, the powder having a sparingly water-soluble property (A) is believed to be attached to and spread in the surrounding area of the openings of dentinal tubules, thereby being able to seal the openings of dentinal tubules effectively. Note that the powder having a sparingly water-soluble property (A) of the present invention is in the state of a dispersed powder in the toothpaste composition, preferably is in the state of a dispersed powder having an average particle diameter of from 1 to 20 μm, and more preferably is in the state of a dispersed powder having an average particle diameter of from 2 to 10 μm.

The toothpaste composition for dentin hypersensitivity of the present invention may comprise singly one kind of the powder having a sparingly water-soluble property (A) selected from the fatty acid divalent metal salt (A-1), the fatty acid acyllysine (A-2), and the fatty acid acyltaurine divalent metal salt (A-3), or may contain two or more kinds thereof in combination. The total content of the powder having a sparingly water-soluble property (A) in the toothpaste composition for dentin hypersensitivity of the present invention is preferably from 0.1 to 2 mass %, more preferably from 0.2 to 1.5 mass %, even more preferably from 0.4 to 1.2 mass %, from the viewpoint of ensuring good sealability of the openings of dentinal tubules and good taste of the toothpaste composition.

The fatty acid divalent metal salt, which is the component (A-1), is derived from a saturated fatty acid having from 12 to 18 carbon atoms and is formed of two saturated fatty acids and a divalent metal. Specific examples of such saturated fatty acid include lauric acid, myristic acid, pentadecylic acid, palmitic acid, margaric acid, and stearic acid. Of those, lauric acid, myristic acid, palmitic acid, and stearic acid are preferred, and lauric acid, myristic acid, and stearic acid are more preferred. The two saturated fatty acids forming the component (A-1) are preferably identical to each other. In addition, specific examples of the divalent metal include zinc, calcium, magnesium, strontium, and barium. Of those, zinc, calcium, and magnesium are preferred, and zinc and calcium are more preferred from the viewpoints of intraoral applicability, spreadability, and cost.

The fatty acid acyllysine, which is the component (A-2) and has an acyl group derived from a saturated fatty acid having 12 to 18 carbon atoms, includes, in its molecule, an acyl group derived from such a saturated fatty acid having from 12 to 18 carbon atoms as described above, and an acyllysine that has an acyl group derived from an N-saturated fatty acid is particularly preferred. Examples of such fatty acid acyllysine (A-2) include N-lauroyl lysine, N-myristoyl lysine, N-palmitoyl lysine, and N-stearoyl lysine. Of those, N-lauroyl lysine which has an excellent feel is preferred. As the N-lauroyl lysine, a commercially available product as Nε-lauroyl-L-lysine can be obtained under the trade name of AMIHOPE LL (AJINOMOTO CO., INC.) or the like.

The fatty acid acyltaurine divalent metal salt, which is the component (A-3) and has an acyl group derived from a saturated fatty acid having from 12 to 18 carbon atoms, includes, in its molecule, a fatty acid acyltaurine formed of an acyl group derived from such a saturated fatty acid having from 12 to 18 carbon atoms as described above and a taurine group. An N-saturated fatty acid acyltaurine is preferred as the fatty acid acyltaurine. Examples of such fatty acid acyltaurine include N-lauroyltaurine, N-myristoyltaurine, N-palmitoyltaurine, and N-stearoyltaurine. Of those, an N-lauroyltaurine is preferred from the viewpoints of feel upon use and sealability of the openings of dentinal tubules. As such divalent metal in the fatty acid acyltaurine divalent metal salt (A-3), calcium, zinc, magnesium, and the like are preferred, zinc and calcium are preferred from the viewpoints of feel upon use and sealability of the openings of dentinal tubules, and calcium is more preferred.

The toothpaste composition for dentin hypersensitivity of the present invention comprises a desensitizing agent (B) selected from potassium nitrate, potassium citrate, strontium chloride, and strontium acetate. The desensitizing agent (B) is a component that has the action of changing the balance of ions existing in the surrounding area of a sensory nerve, thereby removing the hypersensitivity of the nerve or making the nerve insensitive, and can also be called a nerve-desensitizing component. Potassium nitrate, potassium citrate, strontium chloride, and strontium acetate are soluble in water, and each of the components in the composition supplies potassium ions or strontium ions, thereby being able to change the balance of ions existing in the surrounding area of a sensory nerve. It is preferred to use, as the desensitizing agent (B), potassium nitrate, strontium chloride, and strontium acetate, from the viewpoint of improving the action of making a nerve insensitive.

The content of the desensitizing agent (B) to be used in the present invention is preferably from 0.04 to 0.06 mole in 100 parts by mass of the toothpaste composition for dentin hypersensitivity of the present invention, or is preferably from 4 to 15 mass %, from 4 to 12 mass % in 100 mass % of the toothpaste composition for dentin hypersensitivity of the present invention, from the viewpoint of alleviating pain attributed to dentin hypersensitivity by effectively changing the balance of ions existing in the surrounding area of a sensory nerve with the desensitizing agent (B) at the time of applying the toothpaste composition in an oral cavity.

In the toothpaste composition for dentin hypersensitivity of the present invention, the mass ratio (A/B) of the component (A) to the component (B) is from 0.02 to 0.5, preferably from 0.04 to 0.5, more preferably from 0.04 to 0.3, even more preferably from 0.08 to 0.3, from the viewpoints of sealing each dentinal tubule with the component (A) to instantaneously prevent pain attributed to dentin hypersensitivity and sustaining the effect while allowing the desensitizing agent (B) to exert a nerve-desensitizing action.

The toothpaste composition for dentin hypersensitivity of the present invention comprises water (C). Thus, the component (B) dissolves in the water and effectively supplies potassium ions or strontium ions, thereby effectively exerting a nerve-desensitizing action, and the poorly water-soluble powder, which is the component (A), can be dispersed and spread. From the viewpoints described above, the content of the water (C) in the toothpaste composition of the present invention is preferably from 10 to 55 mass %, more preferably from 12 to 50 mass %.

Note that the water content (content of water (C)) in the toothpaste composition can be calculated on the basis of the content of water contained in the composition and the water content contained in the components, and can also be measured with, for example, a Karl Fischer moisture titrator. For example, a trace level moisture titrator (Hiranuma Sangyo Co., Ltd.) can be used as the Karl Fischer moisture titrator. 5 g of the toothpaste composition are suspended in 25 g of anhydrous methanol, yielding a suspension, and the water content of the toothpaste composition can be measured with 0.02 g of the suspension by using the titrator.

The toothpaste composition for dentin hypersensitivity of the present invention preferably further comprises a binder (D). As the binder, there may be used one kind of binder or two or more kinds of binders selected from the group consisting of, for example, sodium alginate, sodium carboxymethyl cellulose having a degree of etherification of from 0.7 to 2.0, carageenan, xanthan gum, sodium polyacrylate, hydroxyethyl cellulose, hydroxypropyl cellulose, pectin, gum tragacanth, gum arabic, guar gum, gum karaya, locust bean gum, gellan gum, tamarind gum, psyllium seed gum, polyvinyl alcohol, sodium chondroitin sulfate, and a methoxyethylene-maleic anhydride copolymer. Of those, sodium carboxymethyl cellulose having a degree of etherification of 0.7 to 2.0, carageenan, and xanthan gum are preferred. Further, two or more kinds or three or more kinds selected from the foregoing can be used from the viewpoints of shape-retaining property, stringiness property, and feel upon use. The content of the binder (D) in the toothpaste composition for dentin hypersensitivity of the present invention is preferably from 0.1 to 3 mass %, more preferably from 0.2 to 2 mass %, even more preferably from 0.5 to 1.7 mass %. Note that sodium carboxymethyl cellulose contained as the binder (D) has a degree of etherification of generally 0.7 or more, preferably from 0.7 to 2.0, more preferably from 1.0 to 1.5 from the viewpoint of providing a moderate viscosity.

Note that the toothpaste composition for dentin hypersensitivity of the present invention has a viscosity of preferably from 1,000 to 7,000 dPa·s, more preferably from 1,500 to 5,000 dPa·s at 25° C., from the viewpoint of ensuring good shape-retaining property and good feeling of the use necessary to apply the resultant toothpaste to brushing with a toothbrush. The viscosity of the toothpaste composition of the present invention is preferably obtained by measuring the viscosity of a toothpaste composition three or more days later after the production thereof, and can be measured by using a Helipath viscometer (VISCOMETER TVB-10, Toki Sangyo Co., Ltd.) under the conditions of Rotor T-C at a rotation speed of from 2.5 rpm for 1 minute.

The toothpaste composition for dentin hypersensitivity of the present invention may further comprise any other component for improving dentin hypersensitivity such as a fluoride including sodium monofluorophosphate and a fluoride ion-supplying compound such as tin fluoride, sodium fluoride, and ammonium fluoride, aluminum lactate, calcium phosphate, hydroxyapatite, or arginine-calcium carbonate as long as the effects of the present invention are not impaired.

The toothpaste composition for dentin hypersensitivity of the present invention preferably further comprises powdered cellulose, or carboxymethyl cellulose having a degree of etherification of from 0.1 to 0.6 or a salt thereof (E), from the viewpoint of spreading the powder having a sparingly water-soluble property (A) to seal the openings of dentinal tubules more effectively. Hereinafter, among the members of the component (E), the powdered cellulose is referred to as the component (E-1) and the carboxymethyl cellulose having a degree of etherification of from 0.1 to 0.6 or a salt thereof is referred to as the component (E-2).

The powdered cellulose (E-1) has an average degree of polymerization of from 440 to 2,250, and hence in comparison to crystalline cellulose having an average degree of polymerization of 350 or less, the powdered cellulose not only has a different degree of polymerization and a different degree of crystallization but also has remarkably different physical properties. For example, the crystalline cellulose has moderate abrasive property as understood from the fact that it is used as a peeling agent or the like, but the powdered cellulose hardly has an abrasive property. Further, the powdered cellulose (E-1) is difficult to dissolve in water but has a water absorption property, thus exerting a water retention effect. The powdered cellulose (E-1) retaining water therein has moderate flexibility (softness), and hence it carries the powder having a sparingly water-soluble property (A) via each bristle tip of a toothbrush at the time of brushing, suppressing the load force of each bristle tip of the toothbrush or the like from scattering in various directions. Thus, it is believed that the load force can be applied to the powder having a sparingly water-soluble property (A) efficiently and the powder (A) can be spread well.

The carboxymethyl cellulose having a degree of etherification of from 0.1 to 0.6 or a salt thereof (E-2) is difficult to dissolve in water but has a water absorption property, and thus swells in the presence of water, thereby being able to exert moderate flexibility. Further, the carboxymethyl cellulose or a salt thereof (E-2) does not have a viscosity-increasing function unlike carboxymethyl cellulose or a salt thereof used as a binder and having a degree of etherification outside the above-mentioned range. Further, the carboxymethyl cellulose or a salt thereof (E-2) has the viscosity of a 1% aqueous solution thereof of preferably less than 200 mPa·s, more preferably from 1 to 150 mPa·s at 25° C., even more preferably from 10 to 150 mPa·s. Examples of commercially available products of the carboxymethyl cellulose or a salt thereof (E-2) include SUNROSE SLD-Fl and SUNROSE SLD-FM manufactured by Nippon Paper Chemicals Co., Ltd.

The carboxymethyl cellulose or a salt thereof (E-2) as described above exists in the space between each bristle tip of a toothbrush or the like and the dentinal surface, and carries the powder having a sparingly water-soluble property (A). As a result, while the carboxymethyl cellulose or a salt thereof (E-2) suppresses the load force of each bristle tip of the toothbrush or the like from scattering in various directions at the time of brushing, the load force can be applied to the powder having a sparingly water-soluble property (A) efficiently and the powder (A) can be spread well.

The carboxymethyl cellulose or a salt thereof (E-2) has a degree of etherification of more preferably from 0.1 to 0.5, even more preferably from 0.2 to 0.4. Herein, the degree of etherification refers to the degree of substitution of carboxymethyl groups per glucose unit. The degree of etherification can be obtained in accordance with, for example, the CMC Kogyokai (Association) analysis method (ashing method). 1 g of sodium carboxymethyl cellulose is accurately weighed, is fed into a porcelain crucible, and is fired to ash at 600° C. The resultant sodium oxide is subjected to titration with N/10 sulfuric acid by using phenolphthalein as an indicator. Then, the titer Y mL for 1 g of sodium carboxymethyl cellulose is substituted into the following equation, thus being able to calculate the degree of etherification.

Degree of etherification=(162×Y)/(10,000−80×Y)

Examples of a salt of the carboxymethyl cellulose (E-2) include an alkali metal salt of sodium, potassium, or the like and an ammonium salt. Of those, sodium carboxymethyl cellulose is preferred from the viewpoints of oral applicability, cost, easy availability, and attempt in satisfactory spreading of the powder having a sparingly water-soluble property (A).

The content of the powdered cellulose (E-1) or the carboxymethyl cellulose or a salt thereof (E-2) in the toothpaste composition for dentin hypersensitivity of the present invention is preferably 0.05 mass % or more, more preferably 0.1 mass % or more, even more preferably 0.2 mass % or more, from the following viewpoints of water retention property and moderate flexibility, thus being able to spread the powder having a sparingly water-soluble property (A) to seal dentinal tubules effectively. Further, the content of the component (E-1) or the component (E-2) in the toothpaste composition for dentin hypersensitivity of the present invention is preferably 5 mass % or less, more preferably 2.5 mass % or less, even more preferably 1 mass % or less, even more preferably 0.8 mass % or less, from the following viewpoint of moderate flexibility, thus being able to spread the powder having a sparingly water-soluble property (A) to seal dentinal tubules effectively. Note that one kind of component selected from the component (E-1) and the component (E-2) may be used singly, or two or more kinds thereof may be used in combination.

The average particle diameter of the component (E) is preferably from 10 to 200 μm, more preferably from 15 to 100 μm, even more preferably from 20 to 80 μm, from the viewpoint of allowing the component (E) to exist effectively in the narrow space between each bristle tip of a toothbrush or the like and the dentinal surface at the time of brushing while maintaining its good water retention property.

Further, the mass ratio (A/E) of the component (A), which has a sparingly water-soluble property, to the component (E), which is the powdered cellulose (E-1) or the carboxymethyl cellulose having a degree of etherification of from 0.1 to 0.6 or a salt thereof (E-2), is preferably from 0.02 to 20, more preferably from 0.1 to 10, even more preferably from 0.5 to 7, from the viewpoint that the component (A) is spreadwell while moderate flexibility is exerted by the component (E).

The toothpaste composition for dentin hypersensitivity of the present invention is capable of preventing pain attributed to dentin hypersensitivity by brushing with the toothpaste composition by a usual method. From the viewpoint of preventing the pain attributed to dentin hypersensitivity more effectively, the toothpaste composition is preferably used from 1 to 5 times a day as the method of use for the toothpaste composition, and it is preferred that the use of the toothpaste composition be performed at a frequency of one day or more per month or at a frequency of one to two consecutive weeks.

The total content of granules having a collapse strength of 15 g/granule or more and powders or particles having a high abrasive property, such as crystalline cellulose, in the toothpaste composition for dentin hypersensitivity of the present invention is preferably 2 mass % or less, more preferably 1 mass % or less, even more preferably 0.5 mass % or less, and the toothpaste composition is even more preferably substantially free of granules, powders or particles (does not contain them except in the case where they are inevitably contained in other components of the toothpaste composition), from the viewpoint of not stimulating physically the surface of each dentinal tubule and the viewpoint of preventing the detachment of the powder having a sparingly water-soluble property (A) that has sealed the openings of the dentinal tubules. Note that the collapse strength means a value represented by the average value of the values obtained by measuring the strength of from 10 to 20 granules each having a particle diameter of from 180 to 200 μm by using an ultra micro hardness tester (Shimadzu Corporation, MCTM-500). Further, the collapse strength refers to the strength of granules in a wet state contained in the toothpaste composition.

The toothpaste composition for dentin hypersensitivity of the present invention may contain an abrasive as long as the effects of the present invention are not affected. Examples of the abrasive include calcium phosphate, calcium hydrogen phosphate, calcium carbonate, aluminum hydroxide, aluminum silicate, zirconium silicate, calcium pyrophosphate, and silicic anhydride (abrasive silica: having an oil absorption of from 50 to 150 mL/100 g measured by a method in accordance with JIS K5101-13-2). An abrasive having a radioactive dentin abrasion value (RDA value, which is a value obtained by measurement in accordance with IS011609, Test method for abrasive property, Attachment A) of from 20 to 250 is generally used as the abrasive. The content of the abrasive in the toothpaste composition for dentin hypersensitivity of the present invention is preferably from 0 to 20 mass %, more preferably 15 mass % or less, even more preferably 12 mass % or less. The content of an abrasive having an RDA value of more than 250 in the toothpaste composition for dentin hypersensitivity of the present invention is preferably 2 mass % or less, more preferably 1 mass % or less, even more preferably 0.1 mass % or less, and the toothpaste composition is even more preferably substantially free of the abrasive (does not contain it except in the case where it is inevitably contained in other components of the toothpaste composition).

The toothpaste composition of the present invention is
<1> a toothpaste composition for dentin hypersensitivity which comprises (A) a powder having a sparingly water-soluble property selected from a fatty acid divalent metal salt (A-1) derived from a saturated fatty acid having from 12 to 18 carbon atoms, a fatty acid acyllysine (A-2) having an acyl group derived from a saturated fatty acid having from 12 to 18 carbon atoms, and a fatty acid acyltaurine divalent metal salt (A-3) having an acyl group derived from a saturated fatty acid having from 12 to 18 carbon atoms, (B) a desensitizing agent selected from potassium nitrate, potassium citrate, strontium chloride, and strontium acetate, and (C) water, in which a mass ratio ((A)/(B)) of the component (A) to the component (B) is from 0.02 to 0.5.

Further, the present invention is preferably the following toothpaste composition or method, or application:
<2> the toothpaste composition for dentin hypersensitivity according to the above-mentioned item <1>, in which the content of the component (C) is from 10 to 55 mass %;
<3> the toothpaste composition for dentin hypersensitivity according to the above-mentioned item <1> or <2>, in which the divalent metal of the component (A-1) or the component (A-3) is selected from zinc, calcium, and magnesium;
<4> the toothpaste composition for dentin hypersensitivity according to any one of the above-mentioned items <1> to <3>, in which the divalent metal of the component (A-1) is zinc or calcium;
<5> the toothpaste composition for dentin hypersensitivity according to any one of the above-mentioned items <1> to <4>, in which the component (A-1) is selected from a lauric acid divalent metal salt, a myristic acid divalent metal salt, and a stearic acid divalent metal salt;
<6> the toothpaste composition for dentin hypersensitivity according to any one of the above-mentioned items <1> to <5>, in which the component (A-1) is a stearic acid divalent metal salt;
<7> the toothpaste composition for dentin hypersensitivity according to the above-mentioned item <5> or <6>, in which the divalent metal of the component (A-1) is zinc;
<8> the toothpaste composition for dentin hypersensitivity according to any one of the above-mentioned items <1> to <4>, in which the component (A-1) is a lauric acid divalent metal salt;
<9> the toothpaste composition for dentin hypersensitivity according to the above-mentioned item <8>, in which the divalent metal of the component (A-1) is calcium;

<10> the toothpaste composition for dentin hypersensitivity according to any one of the above-mentioned items <1> to <9>, in which the component (A-2) is selected from N-lauroyl lysine, N-myristoyl lysine, N-palmitoyl lysine, and N-stearoyl lysine;
<11> the toothpaste composition for dentin hypersensitivity according to any one of the above-mentioned items <1> to <10>, in which the component (A-2) is N-lauroyl lysine;
<12> the toothpaste composition for dentin hypersensitivity according to any one of the above-mentioned items <1> to <11>, in which the divalent metal of the component (A-3) is calcium;
<13> the toothpaste composition for dentin hypersensitivity according to any one of the above-mentioned items <1> to <12>, in which the component (A-3) is selected from an N-lauroyltaurine divalent metal salt, an N-myristoyltaurine divalent metal salt, an N-palmitoyltaurine divalent metal salt, and an N-stearoyltaurine divalent metal salt;
<14> the toothpaste composition for dentin hypersensitivity according to any one of the above-mentioned items <1> to <13>, in which the component (A-3) is an N-lauroyltaurine divalent metal salt;

<15> the toothpaste composition for dentin hypersensitivity according to any one of the above-mentioned items <1> to <14>, in which the composition comprises the component (A) in an amount of from 0.1 to 2 mass % and the component <B> in an amount of from 4 to 15 mass %;
<16> the toothpaste composition for dentin hypersensitivity according to any one of the above-mentioned items <1> to <14>, in which the composition comprises the component (A) in an amount of from 0.4 to 1.2 mass % and the component <B> in an amount of from 4 to 12 mass %;
<17> the toothpaste composition for dentin hypersensitivity according to any one of the above-mentioned items <1> to <16>, in which the composition comprises the component (B) in an amount of from 0.04 to 0.06 mol;
<18> the toothpaste composition for dentin hypersensitivity according to any one of the above-mentioned items <1> to <17>, in which the mass ratio ((A)/(B)) of the component (A) to the component (B) is from 0.02 to 0.5, preferably from 0.04 to 0.5, more preferably from 0.04 to 0.3;
<19> the toothpaste composition for dentin hypersensitivity according to any one of the above-mentioned items <1> to <17>, in which the mass ratio ((A)/(B)) of the component (A) to the component (B) is from 0.08 to 0.25;

<20> the toothpaste composition for dentin hypersensitivity according to any one of the above-mentioned items <1> to <19>, in which the composition further comprises (D) a binder in an amount of from 0.1 to 3 mass %;
<21> the toothpaste composition for dentin hypersensitivity according to any one of the above-mentioned items <1> to <20>, in which the composition further comprises (E) powdered cellulose, or carboxymethyl cellulose having a degree of etherification of from 0.1 to 0.6 or a salt thereof;
<22> the toothpaste composition for dentin hypersensitivity according to the above-mentioned item <21>, in which the composition comprises the component (E) in an amount of from 0.05 to 5 mass %;
<23> the toothpaste composition for dentin hypersensitivity according to the above-mentioned item <21> or <22>, in which amass ratio of the component (A) to the component (E) is from 0.02 to 20;

<24> the toothpaste composition for dentin hypersensitivity according to any one of the above-mentioned items <1> to <23>, in which the content of granules having a collapse strength of 15 g/granule or more is 2 mass % or less;
<25> the toothpaste composition for dentin hypersensitivity according to any one of the above-mentioned items <1> to <24>, in which the content of an abrasive having an RDA value of from 25 to 250 is 15 mass % or less;
<26> the toothpaste composition for dentin hypersensitivity according to any one of the above-mentioned items <1> to <25>, in which the content of an abrasive having an RDA value of more than 250 is 2 mass % or less;
<27> the toothpaste composition for dentin hypersensitivity according to any one of the above-mentioned items <1> to <26>, in which the toothpaste composition for dentin hypersensitivity has a viscosity of from 1,000 to 7,000 dPa·s at 25° C.;

<28> use of the toothpaste composition for dentin hypersensitivity according to anyone of the above-mentioned items <1> to <27> for use in preventing pain attributed to dentin hypersensitivity;
<29> use of the toothpaste composition for dentin hypersensitivity according to any one of the above-mentioned items <1> to <27> for producing a preventive agent for dentin hypersensitivity; and
<30> a method of preventing dentin hypersensitivity, comprising applying the toothpaste composition for dentin hypersensitivity according to any one of the above-mentioned items <1> to <27> in an oral cavity.

### EXAMPLES

Hereinafter, the present invention is specifically described by way of Examples. Note that the content of each component is represented in a mass % unit unless otherwise specifically stated in tables.

### Examples 1 to 9 and Comparative Examples 1 to 3

Toothpaste compositions having compositions shown in Table 1 and Table 2 were each prepared in accordance with a usual method. Each toothpaste composition obtained was used to perform the evaluation shown below of the sealed state of openings of dentinal tubules. Further, each toothpaste composition having a composition shown in Table 1 was used to perform the evaluation of the effect of alleviating pain attributed to dentin hypersensitivity immediately after the use thereof and two hours later after the use thereof. Each toothpaste composition having a composition shown in Table 2 was used to perform the evaluation of the effect of alleviating pain attributed to dentin hypersensitivity after two-week of continuous use thereof. Table 1 and Table 2 each show the results of the evaluation. Note that the viscosity of the toothpaste composition prepared in each of Examples 1 to 9 and Comparative Examples 1 to 3 was adjusted so as to be from 3,500 to 4, 500 dPa·s at 25° C. one month after the preparation. The viscosity of the toothpaste composition prepared in each of Examples 1 to 4 was 3,800 dPa·s (measurement conditions: a Helipath viscometer, VISCOMETER TVB-10 (Toki Sangyo Co., Ltd.), Rotor T-C at a rotation speed of 2.5 rpm for 1 minute).

### Evaluation of Sealed State of Openings of Dentinal Tubules

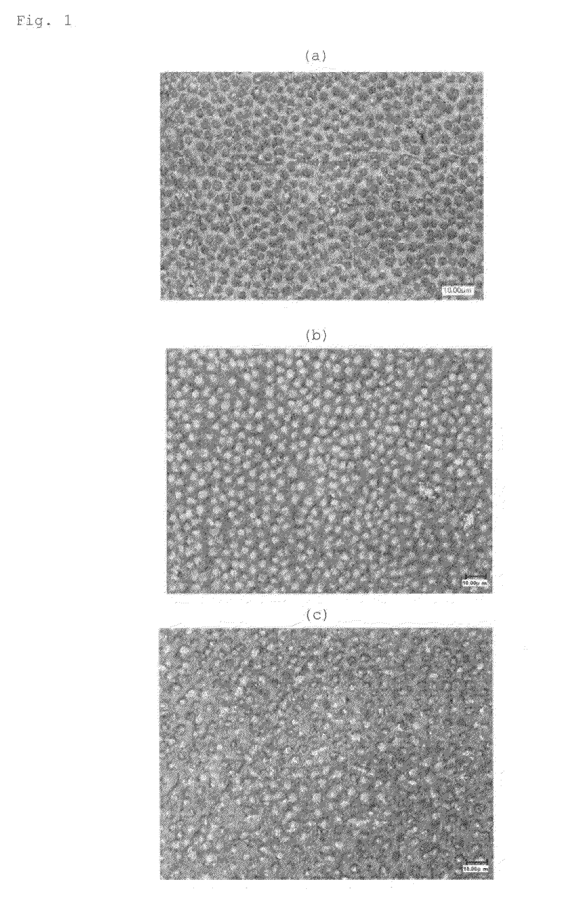

Dentins of bovine teeth were used as samples. The samples were subjected to demineralization in a 0.5% phosphoric acid aqueous solution at room temperature for 30 minutes. The surfaces of the demineralized samples were subjected to polishing treatment with a sandpaper having an abrasive grain size of 40 μm and a sandpaper having an abrasive grain size of 3 μm. The surface of each of the demineralized samples was subjected to reciprocating brushing five times with a toothbrush (Deep Clean Super Compact Normal, manufactured by Kao Corporation) by using 1 g of the toothpaste composition prepared in each of Examples and Comparative Examples, followed by the washing of the sample with distilled water for 30 seconds. A digital microscope (VHX-1000 manufactured by Keyence Corporation) was used to observe, at a magnification of 2,000, the openings of dentinal tubules in the surface of each of the washed samples, thereby visually confirming the state of the openings of the dentinal tubules. FIG. 1(a) is a micrograph showing the state of a sample treated with the toothpaste composition of Comparative Example 2. As shown in FIG. 1(a), almost all openings of dentinal tubules are black, and hence almost all the openings are found to be open. FIG. 1(b) is a micrograph showing the state of a sample treated with the toothpaste composition of Example 8. As shown in FIG. 1(b), almost all openings of dentinal tubules are white, and hence a state in which almost all the openings are sealed with the toothpaste composition is found. FIG. 1(c) is a micrograph showing the state of a sample treated with the toothpaste composition of Example 1. As shown in FIG. 1(c), a state in which some parts of openings of dentinal tubules are open, but most openings are sealed, is found.

For the evaluation of the sealed state of the openings of dentinal tubules by using the toothpaste composition, the state shown in FIG. 1(a) was represented by “C,” the state shown in FIG. 1(b) was represented by “AA,” the state shown in FIG. 1(c) showing that some openings of dentinal tubules were open, but most openings thereof were sealed when compared with FIG. 1(b) was represented by “A,” and the state that about half openings of dentinal tubules were sealed was represented by “B.” Table 1 and Table 2 show the results of the evaluation.

### Evaluation 1 of Alleviation of Pain Attributed to Dentin Hypersensitivity

The toothpaste composition prepared in each of Examples 1 to 7, and Comparative Examples 1 and 2 in Table 1 was used to perform the evaluation of the degree of the prevention or suppression of pain attributed to hypersensitive dentin immediately after the use thereof and two hours after the use thereof. Persons whose teeth hurt every day in the recent one month or persons whose teeth hurt several times a week volunteered as panelists serving as evaluation subjects. The following evaluation method was adopted. Three panelists used each toothpaste composition, held cold water in their mouth immediately after the use thereof and two hours after the use thereof, and evaluated for pain attributed to dentin hypersensitivity. The evaluation was performed on the basis of their answers selected from three kinds, i.e., “3: teeth do not hurt,” “2: teeth hurt slightly,” and “1: teeth hurt.” Each of the total values of the answers from the panelists is shown in Table 1 as the evaluation of pain attributed to dentin hypersensitivity. An evaluation with a larger total value means that pain attributed to dentin hypersensitivity is suppressed or reduced.

### Evaluation 2 of Alleviation of Pain Attributed to Dentin Hypersensitivity

The toothpaste composition prepared in each of Examples 8 and 9, and Comparative Example 3 in Table 2 was used to perform the evaluation of the degree of the prevention or suppression of pain attributed to dentin hypersensitivity by long-term use thereof. Persons whose teeth hurt every day in the recent one month or persons whose teeth hurt several times a week volunteered as panelists serving as evaluation subjects. After eleven panelists used each toothpaste composition three times a day for two weeks, evaluation was performed on whether pain attributed to dentin hypersensitivity was prevented or suppressed. The evaluation was performed after two-week of continuous use of each toothpaste composition on the basis of their answers concerning “whether you feel free from hurting” selected from “3: I think so,” “2: I slightly think so,” and “1: I do not think so or I am not sure”. Each of the total values of the answers from the panelists is shown in Table 2 as the evaluation of pain attributed to dentin hypersensitivity. An evaluation with a larger total value means that pain attributed to dentin hypersensitivity is suppressed or reduced.

TABLE 1

| | | Example | Example | Example | Example | Comparative | Example | Comparative | Example | Example |
|---|---|---|---|---|---|---|---|---|---|---|
| | | 1 | 2 | 3 | 4 | Example 1 | 5 | Example 2 | 6 | 7 |
| A | Zinc stearate | 1.0 | — | — | — | — | 1.0 | — | 0.2 | 0.5 |
| | Calcium laurate | — | 1.0 | — | — | — | — | — | — | — |
| | Nε-lauroyl-L-lysine *1 | — | — | 1.0 | — | — | — | — | — | — |
| | N-lauroyltaurine calcium | — | — | — | 1.0 | — | — | — | — | — |
| | Zinc oxide | — | — | — | — | 1.0 | — | — | — | — |
| B | Potassium nitrate | 5.0 | 5.0 | 5.0 | 5.0 | 5.0 | — | 5.0 | 5.0 | 5.0 |
| | Strontium chloride | — | — | — | — | — | 10.0 | — | — | — |
| | Silicic anhydride A *2 | 7.0 | 7.0 | 7.0 | 7.0 | 7.0 | 7.0 | 7.0 | 7.0 | 7.0 |
| | Silicic anhydride B *3 | 8.0 | 8.0 | 8.0 | 8.0 | 8.0 | 8.0 | 8.0 | 8.0 | 8.0 |
| D | Sodium carboxymethyl | 1.2 | 1.2 | 1.2 | 1.2 | 1.2 | 1.2 | 1.2 | 1.2 | 1.2 |
| | cellulose X *4 | | | | | | | | | |
| | Polyethylene glycol | 5.0 | 5.0 | 5.0 | 5.0 | 5.0 | 5.0 | 5.0 | 5.0 | 5.0 |
| | Sorbitol (70%) | 50 | 50 | 50 | 50 | 50 | 50 | 50 | 50 | 50 |
| | Saccharin sodium | 0.2 | 0.2 | 0.2 | 0.2 | 0.2 | 0.2 | 0.2 | 0.2 | 0.2 |
| | Flavor | 0.8 | 0.8 | 0.8 | 0.8 | 0.8 | 0.8 | 0.8 | 0.8 | 0.8 |
| | Sodium lauroylmethyl | 0.7 | 0.7 | 0.7 | 0.7 | 0.7 | 0.7 | 0.7 | 0.7 | 0.7 |
| | taurate | | | | | | | | | |
| | 48% NaOH | 0.2 | 0.2 | 0.2 | 0.2 | 0.2 | 0.2 | 0.2 | 0.2 | 0.2 |
| | Purified water | Balance | Balance | Balance | Balance | Balance | Balance | Balance | Balance | Balance |
| Total | | 100 | 100 | 100 | 100 | 100 | 100 | 100 | 100 | 100 |
| C | Water (water content) | 36.0 | 36.0 | 36.0 | 36.0 | 36.0 | 31.0 | 37.0 | 36.8 | 36.5 |

| | | | | | | | | | |
|---|---|---|---|---|---|---|---|---|---|
| A/B | 0.2 | 0.2 | 0.2 | 0.2 | — | 0.1 | — | 0.04 | 0.1 |
| State of dentinal tubules | A | A | A | A | C | A | C | B | A |
| Evaluation of pain immediately | 8 | 7 | 9 | 7 | 4 | 9 | 4 | 6 | 8 |
| after use | | | | | | | | | |
| Evaluation of pain two hours | 7 | 5 | 8 | 6 | 3 | 7 | 3 | 4 | 7 |
| after use | | | | | | | | | |

*1: AMIHOPE LL (AJINOMOTO CO., INC.)
*2: Sylopure 25 (FUJI SILYSIA CHEMICAL LTD.) oil absorption: 310 ml/100 g
*3: SORBOSIL AC77 (PT PQ Sillicas Indonesia) RDA value: 125
*4: SUNROSE F35SH (Nippon Paper Chemicals Co., Ltd.) degree of etherification: from 1.0 to 1.15

TABLE 2

| | | | Comparative | |
|---|---|---|---|---|
| | | Example 8 | Example 3 | Example 9 |
| A | Zinc stearate | 1.0 | — | 1.0 |
| B | Potassium nitrate | 5.0 | 5.0 | — |
| | Strontium chloride | — | — | 6.0 |
| | Powdered cellulose *5 | 0.5 | 0.5 | 0.5 |
| | Silicic anhydride A *2 | 7 | 7 | 7 |
| | Silicic anhydride B *3 | 8 | 8 | 8 |
| D | Sodium carboxymethyl | 1.2 | 1.2 | 1.2 |
| | cellulose X *4 | | | |
| | Polyethylene glycol | 5 | 5 | 5 |
| | Sorbitol (70%) | 50 | 50 | 50 |
| | Saccharin sodium | 0.2 | 0.2 | 0.2 |
| | Flavor | 0.8 | 0.8 | 0.8 |
| | Sodium lauroylmethyl taurate | 0.7 | 0.7 | 0.7 |
| | 48% NaOH | 0.2 | 0.2 | 0.2 |
| | Purified water | Balance | Balance | Balance |
| | Total | 100 | 100 | 100 |
| C | Water (water content) | 35.7 | 36.7 | 34.7 |

| | | | |
|---|---|---|---|
| A/B | 0.2 | — | 0.17 |
| State of dentinal tubules | AA | C | AA |
| Evaluation of pain attributed to | 27 | 16 | 28 |
| dentin hypersensitivity | | | |

*2: Sylopure 25 (FUJI SILYSIA CHEMICAL LTD.) oil absorption: 310 ml/100 g
*3: SORBOSIL AC77 (PT PQ Sillicas Indonesia) RDA value: 125
*4: SUNROSE F35SH (Nippon Paper Chemicals Co., Ltd.) degree of etherification: 1.0 to 1.15
*5: KC FLOCK W-400 (Nippon Paper Chemicals Co., Ltd.) average particle diameter: 24 μm

As shown in Table 1, when the toothpaste composition of each of Examples 1 to 7 containing the powder having a sparingly water-soluble property (A) and the desensitizing agent (B) of the present invention was used, it was found that pain attributed to dentin hypersensitivity was alleviated immediately after the use thereof and two hours after the use thereof. On the other hand, when the toothpaste composition of each of Comparative Examples 1 and 2 not containing the powder having a sparingly water-soluble property (A) was used, the resultant evaluation results showed that some panelists felt that pain attributed to dentin hypersensitivity was slightly alleviated immediately after the use thereof, but pain attributed to dentin hypersensitivity was felt by them two hours after the use thereof, and hence no sustained effect thereof was confirmed. Further, when the toothpaste composition of each of Examples 1 to 7 of the present invention was used, it was found that most openings of dentinal tubules were sealed or about half openings of dentinal tubules were sealed. On the other hand, when the toothpaste composition of each of Comparative Examples 1 and 2 was used, it was found that nearly no openings of dentinal tubules were sealed.

As shown in Table 2, when the toothpaste composition of each of Examples 8 and 9 comprising the poorly water-soluble powder (A) and the desensitizing agent (B) of the present invention was used, the resultant evaluation results showed that long-term use of each toothpaste composition suppressed pain of most panelists attributed to dentin hypersensitivity. On the other hand, when the toothpaste composition of Comparative Example 3 was used, the result of the evaluation by a few panelists was that even long-term use of the toothpaste composition only slightly alleviated their pain attributed to dentin hypersensitivity. Further, when the toothpaste composition of each of Examples 8 and 9 of the present invention was used, it was found that almost all openings of dentinal tubules were sealed. When the toothpaste composition of Comparative Example 3 was used, it was found that almost all openings of dentinal tubules were in an open state (not sealed).

### Examples 10 to 15

Each toothpaste composition having composition shown in Table 3 was prepared in accordance with a usual method. Each toothpaste composition obtained was used to perform the evaluation described above of the sealed state of openings of dentinal tubules. Table 3 shows the evaluation results of the sealed state of openings of dentinal tubules.

TABLE 3

| | | Example | Example | Example | Example | Example | Example |
|---|---|---|---|---|---|---|---|
| | | 10 | 11 | 12 | 13 | 14 | 15 |
| A | Zinc stearate | 0.5 | — | — | — | — | 0.5 |
| | Calcium laurate | — | 0.5 | — | — | — | — |
| | Nε-lauroyl-L-lysine *1 | — | — | 0.5 | — | — | — |
| | N-lauroyltaurine calcium | — | — | — | 0.5 | — | — |
| | Zinc oxide | — | — | — | — | 0.5 | — |
| B | Potassium nitrate | 5.0 | 5.0 | 5.0 | 5.0 | 5.0 | 5.0 |
| | Powdered cellulose *5 | 0.5 | 0.5 | 0.5 | 0.5 | — | 0.1 |
| | Sodium carboxymethyl | — | — | — | — | 0.5 | — |
| | cellulose Y *6 | | | | | | |
| | Silicic anhydride A *2 | 7.0 | 7.0 | 7.0 | 7.0 | 7.0 | 7.0 |
| | Silicic anhydride B *3 | 8.0 | 8.0 | 8.0 | 8.0 | 8.0 | 8.0 |
| D | Sodium carboxymethyl | 1.2 | 1.2 | 1.2 | 1.2 | 1.2 | 1.2 |
| | cellulose X *4 | | | | | | |
| | Polyethylene glycol | 5.0 | 5.0 | 5.0 | 5.0 | 5.0 | 5.0 |
| | Sorbitol (70%) | 50 | 50 | 50 | 50 | 50 | 50 |
| | Saccharin sodium | 0.2 | 0.2 | 0.2 | 0.2 | 0.2 | 0.2 |
| | Flavor | 0.8 | 0.8 | 0.8 | 0.8 | 0.8 | 0.8 |
| | Sodium lauroylmethyl | 0.7 | 0.7 | 0.7 | 0.7 | 0.7 | 0.7 |
| | taurate | | | | | | |
| | 48% NaOH | 0.2 | 0.2 | 0.2 | 0.2 | 0.2 | 0.2 |
| | Purified water | Balance | Balance | Balance | Balance | Balance | Balance |
| Total | | 100 | 100 | 100 | 100 | 100 | 100 |
| C | Water (water content) | 36.0 | 36.0 | 36.0 | 36.0 | 36.0 | 36.4 |

| | | | | | | |
|---|---|---|---|---|---|---|
| A/B | 0.1 | 0.1 | 0.1 | 0.1 | 0.1 | 0.1 |
| State of dentinal tubules | AA | AA | AA | AA | AA | A |

*1: AMIHOPE LL (AJINOMOTO CO., INC.)
*2: Sylopure 25 (FUJI SILYSIA CHEMICAL LTD.) oil absorption: 310 ml/100 g
*3: SORBOSIL AC77 (PT PQ Sillicas Indonesia) RDA value: 125
*4: SUNROSE F35SH (Nippon Paper Chemicals Co., Ltd.) degree of etherification: 1.0 to 1.15
*5: KC FLOCKW-400 (Nippon Paper Chemicals Co ., Ltd.) average particle diameter: 24 μm
*6: SUNROSE SLD-F1 (Nippon Paper Chemicals Co., Ltd.) degree of etherification: 0.2 to 0.3, average particle diameter: 50 to 60 μm

As shown in Table 3, when the toothpaste composition of each of Examples 10 to 15 containing the powder having a sparingly water-soluble property (A) and the desensitizing agent (B) of the present invention, and further containing the component (E) was used, the result that the sealability of openings of dentinal tubules was further improved was obtained. As mentioned above, when the toothpaste composition of each of Examples 10 to 15 having high sealability of openings of dentinal tubules is used, an extremely excellent suppressing effect of pain attributed to hypersensitivity is believed to be exerted, as those of Examples 8 and 9 in Table 2.

Each toothpaste composition having a composition shown in Table 4 was prepared in accordance with a usual method. Each toothpaste composition obtained was used to perform the evaluation described above of the sealed state of openings of dentinal tubules. Table 4 shows the evaluation results of the sealed state of openings of dentinal tubules.

TABLE 4

| | | Example 16 | Example 17 | Example 18 |
|---|---|---|---|---|
| A | Zinc stearate | 1.2 | 1 | 1 |
| B | Potassium nitrate | 5 | 5 | 5 |
| E | Powdered cellulose *5 | 0.8 | 0.5 | 0.5 |
| | Silicic anhydride A *2 | 7 | 7 | 7 |
| | Silicic anhydride B *3 | 8 | 8 | 8 |
| D | Sodium carboxymethyl | 0.3 | 0.6 | 1.3 |
| | cellulose X *4 | | | |
| | Xanthan gum | 0 | 0.2 | 0 |
| | Carageenan | 0 | 0 | 0.2 |
| | Polyethylene glycol | 5 | 5 | 5 |
| | Glycerin | 20 | 20 | 0 |
| | Sorbitol (70%) | 49 | 40 | 30 |
| | Saccharin sodium | 0.2 | 0.2 | 0.2 |
| | Flavor | 0.8 | 0.8 | 0.8 |
| | Sodium lauroylmethyl | 0.7 | 0.7 | 0.7 |
| | taurate | | | |
| | 48% NaOH | 0.2 | 0.2 | 0.2 |
| | Purified water | 1.8 | 10.8 | 40.1 |
| | Total | 100 | 100 | 100 |
| C | Water (water content) | 16.9 | 23.2 | 49.1 |

| | | | |
|---|---|---|---|
| A/B | 0.24 | 0.2 | 0.2 |
| State of dentinal tubules | AA | AA | AA |

*2: Sylopure 25 (FUJI SILYSIA CHEMICAL LTD.) oil absorption: 310 ml/100 g
*3: SORBOSIL AC77 (PT PQ Sillicas Indonesia) RDA value: 125
*4: SUNROSE F35SH (Nippon Paper Chemicals Co., Ltd.): degree of etherification: 1.0 to 1.15
*5: KC FLOCK W-400 (Nippon Paper Chemicals Co., Ltd.): average particle diameter: 24 μm

As shown in Table 4, when the toothpaste composition of each of Examples 16 to 18 comprising the powder having a sparingly water-soluble property (A) and the desensitizing agent (B) of the present invention, and further comprising the component (E) was used, the result that the sealability of openings of dentinal tubules was further improved was obtained. As mentioned above, when the toothpaste composition of each of Examples 16 to 18 having high sealability of openings of dentinal tubules is used, an extremely excellent suppressing effect of pain attributed to hypersensitivity is believed to be exerted, as those of Examples 8 and 9 in Table 2.

1-14. (canceled)

15. A method of preventing dentin hypersensitivity comprising applying, in an oral cavity, a toothpaste composition for dentin hypersensitivity,
wherein the composition comprises components (A), (B), and (C):
(A) a powder having a sparingly water-soluble property selected from the group consisting of a fatty acid divalent metal salt (A-1) derived from a saturated fatty acid having from 12 to 18 carbon atoms, a fatty acid acyllysine (A-2) having an acyl group derived from a saturated fatty acid having from 12 to 18 carbon atoms, and a fatty acid acyltaurine divalent metal salt (A-3) having an acyl group derived from a saturated fatty acid having from 12 to 18 carbon atoms;
(B) a desensitizing agent selected from the group consisting of potassium nitrate, potassium citrate, strontium chloride, and strontium acetate; and
(C) water;
wherein a mass ratio ((A)/(B)) of component (A) to component (B) is from 0.02 to 0.5.

16. The method of preventing dentin hypersensitivity according to claim 15, wherein a content of component (C) is from 10 to 55 mass %.

17. The method of preventing dentin hypersensitivity according to claim 15, wherein a divalent metal of component (A-1) or component (A-3) is selected from the group consisting of zinc, calcium, and magnesium.

18. The method of preventing dentin hypersensitivity according to claim 15, wherein component (A-2) is selected from the group consisting of N-lauroyl lysine, N-myristoyl lysine, N-palmitoyl lysine, and N-stearoyl lysine.

19. The method of preventing dentin hypersensitivity according to claim 15, wherein the composition comprises component (B) in an amount of from 4 to 15 mass %.

20. The method of preventing dentin hypersensitivity according to claim 15, wherein the composition comprises component (A) in an amount of from 0.1 to 2 mass % and component (B) in an amount of from 4 to 15 mass %.

21. The method of preventing dentin hypersensitivity according to claim 15, wherein the composition further comprises (D) a binder in an amount of from 0.1 to 3 mass %.

22. The method of preventing dentin hypersensitivity according to claim 15, wherein the composition further comprises (E) powdered cellulose, or carboxymethyl cellulose having a degree of etherification of from 0.1 to 0.6 or a salt thereof.

23. The method of preventing dentin hypersensitivity according to claim 22, wherein the composition comprises component (E) in an amount of from 0.05 to 5 mass %.

24. The method of preventing dentin hypersensitivity according to claim 22, wherein a mass ratio ((A)/(E)) of component (A) to component (E) is from 0.02 to 20.

25. A toothpaste composition for dentin hypersensitivity, comprising components (A), (B), (C), and (E):
(A) a powder having a sparingly water-soluble property selected from the group consisting of a fatty acid divalent metal salt (A-1) derived from a saturated fatty acid having from 12 to 18 carbon atoms, a fatty acid acyllysine (A-2) having an acyl group derived from a saturated fatty acid having from 12 to 18 carbon atoms, and a fatty acid acyltaurine divalent metal salt (A-3) having an acyl group derived from a saturated fatty acid having from 12 to 18 carbon atoms;
(B) a desensitizing agent selected from the group consisting of potassium nitrate, potassium citrate, strontium chloride, and strontium acetate;
(C) water; and
(E) powdered cellulose, or carboxymethyl cellulose having a degree of etherification of from 0.1 to 0.6 or a salt thereof
wherein a mass ratio ((A)/(B)) of component (A) to component (B) is from 0.02 to 0.5.

26. The toothpaste composition for dentin hypersensitivity according to claim 25, wherein a content of component (C) is from 10 to 55 mass %.

27. The toothpaste composition for dentin hypersensitivity according to claim 25, wherein a divalent metal of component (A-1) or component (A-3) is selected from the group consisting of zinc, calcium, and magnesium and component (A-2) is selected from the group consisting of N-lauroyl lysine, N-myristoyl lysine, N-palmitoyl lysine, and N-stearoyl lysine.

28. The toothpaste composition for dentin hypersensitivity according to claim 25, wherein the composition comprises component (A) in an amount of from 0.1 to 2 mass % and component (B) in an amount of 4 to 15 mass %.

29. The toothpaste composition for dentin hypersensitivity according to claim 25, wherein the composition further comprises (D) a binder in an amount of from 0.1 to 3 mass %.

30. The toothpaste composition for dentin hypersensitivity according to claim 25, wherein the composition comprises component (E) in an amount of from 0.05 to 5 mass %.

31. The toothpaste composition for dentin hypersensitivity according to claim 25, wherein a mass ratio ((A)/(E)) of component (A) to component (E) is from 0.02 to 20.

32. The toothpaste composition for dentin hypersensitivity according to claim 25, wherein the composition has a viscosity of from 1,000 to 7,000 dPa·s at 25° C.

33. The toothpaste composition for dentin hypersensitivity according to claim 25, wherein the composition comprises an abrasive having an RDA value of from 25 to 250 in an amount of 15 mass % or less.

34. The toothpaste composition for dentin hypersensitivity according to claim 25, wherein the composition comprises an abrasive having an RDA value of more than 250 in an amount of 2 mass % or less.
